SEQUENCE LISTING (16 sequences)

SEQ ID NO: 1 - DNA, 2025 bp, Artificial Sequence
  synthetic
atgagcggtcatcaccatcatcatcacagcggcgtagtatcggaagtagacattgcaaaagccgatccagctgcggccagccacccactgctgctgaatggtgacgcaaccgtggcgcaaaagaatccgggctccgttgcggagaataacctgtgtagccagtatgaagagaaggttcgtccgtgtattgacctgatcgactccctgcgtgcgctgggtgtggagcaggacttggcactgccggctattgcggtcatcggtgatcaatctagcggtaagagcagcgtcctggaggcgctgagcggtgttgcgctgccgcgtggtagcggcattgtcacccgttgccctctggtcttgaaactgaagaaactggtcaatgaagataaatggcgtggcaaagtgagctaccaggattacgaaatcgagatcagcgacgcatccgaggtggaaaaagaaatcaataaagctcagaatgcgatcgcgggtgagggtatgggcattagccacgagctgattaccctggaaatttcgagccgcgatgtcccggacctgaccctgatcgatctgccgggtattacgcgcgtcgcagttggcaaccagccagcggacattggctacaagatcaaaacgctgattaagaagtatatccaacgtcaagaaaccattagcttggttgtggtgccgagcaacgttgacattgcgaccacggaggccctgagcatggcacaagaggttgatccggagggtgaccgcaccatcggcattctgacgaagccggacctggtggataaaggtacggaggacaaagtggtggacgtggttcgcaatctggtttttcacttgaagaaaggttacatgattgttaaatgccgtggtcaacaggagattcaagatcagctgagcctgagcgaggccctgcagcgcgagaaaatcttcttcgagaaccacccgtatttccgtgatctgctggaggaaggtaaggccacggtgccgtgcctggcggagaaactgaccagcgagctgatcacccatatctgcaaatccctgccgctgctggagaaccagatcaaggaaacgcatcagcgtatcaccgaagagttgcagaaatatggtgttgatatcccggaggatgagaacgagaagatgttctttctgattgataagatcaacgctttcaaccaagacattactgcgctgatgcaaggcgaagaaacggttggtgaggaagatattcgtctgttcacccgtctgcgccacgagtttcataagtggagcacgattatcgagaataactttcaagaaggccacaaaatcctgagccgtaagatccaaaagtttgagaaccaataccgtggtcgtgagctgccgggttttgtcaattaccgtacttttgaaaccattgtgaaacagcagattaaggcattggaggaaccggctgtggatatgctgcacaccgtcaccgacatggttcgtctggcctttaccgatgtttccatcaagaacttcgaagagttcttcaatctgcatcgtacggcgaagagcaagattgaggacattcgcgccgaacaggaacgcgagggtgagaagctgatccgcctgcactttcagatggagcagattgtgtactgtcaagatcaagtttaccgtggtgcgctgcagaaagtccgtgaaaaagaactggaagaggaaaagaagaaaaagagctgggatttcggcgcgtttcagagcagctcggcaaccgactcctctatggaagagatttttcaacacttgatggcctatcaccaggaagcttctaagcgtatctccagccacatcccgctgatcatccaattcttcatgttgcagacctatggccagcaactgcaaaaagcgatgctgcagctgctgcaagacaaagacacgtacagctggttgctgaaagaacgcagcgacaccagcgacaaacgcaaatttctgaaggaacgtttggcccgcttgacccaagcacgtcgtcgcctggcacagttcccgggctaactcgag

SEQ ID NO: 2 - protein, 672 aa, Artificial sequence
  synthetic
MSGHHHHHHSGVVSEVDIAKADPAAASHPLLLNGDATVAQKNPGSVAENNLCSQYEEKVRPCIDLIDSLRALGVEQDLALPAIAVIGDQSSGKSSVLEALSGVALPRGSGIVTRCPLVLKLKKLVNEDKWRGKVSYQDYEIEISDASEVEKEINKAQNAIAGEGMGISHELITLEISSRDVPDLTLIDLPGITRVAVGNQPADIGYKIKTLIKKYIQRQETISLVVVPSNVDIATTEALSMAQEVDPEGDRTIGILTKPDLVDKGTEDKVVDVVRNLVFHLKKGYMIVKCRGQQEIQDQLSLSEALQREKIFFENHPYFRDLLEEGKATVPCLAEKLTSELITHICKSLPLLENQIKETHQRITEELQKYGVDIPEDENEKMFFLIDKINAFNQDITALMQGEETVGEEDIRLFTRLRHEFHKWSTIIENNFQEGHKILSRKIQKFENQYRGRELPGFVNYRTFETIVKQQIKALEEPAVDMLHTVTDMVRLAFTDVSIKNFEEFFNLHRTAKSKIEDIRAEQEREGEKLIRLHFQMEQIVYCQDQVYRGALQKVREKELEEEKKKKSWDFGAFQSSSATDSSMEEIFQHLMAYHQEASKRISSHIPLIIQFFMLQTYGQQLQKAMLQLLQDKDTYSWLLKERSDTSDKRKFLKERLARLTQARRRLAQFPG

SEQ ID NO: 3 - DNA, 1521 bp, Artificial sequence
  synthetic
atgagcggtcaccatcatcaccaccatagcggcgtagtaagcgaagtcgatattgcaaaagcggatccggctgcagcgtcgcacccgctgctgctgaatggtgatgcgacggtggcccagaagaatccgggtagcgttgcggagaacaatttgtgtagccagtacgaagagaaagttcgtccgtgcatcgacctgatcgacagcctgcgtgcgctgggcgttgaacaggacctggcactgccggcgatcgcagtcatcggtgatcaatccagcggcaaatcttccgtcctggaagctctgagcggtgtggcgctgccgcgtggcagcggcattgtcacgcgctgcccgttggtgctgaaactgaaaaagctggtgaatgaggacaaatggcgcggtaaggtgtcctaccaggactatgagattgaaattagcgacgctagcgaagttgaaaaagagatcaataaagcgcaaaacgcgattgccggtgagggcatgggtatcagccacgaactgatcacgctggagatcagcagccgcgatgtcccggacctgaccctgattgacctgccgggtatcactcgtgttgcggttggcaatcaaccggccgatattggctacaagattaagaccctgatcaaaaagtatatccagcgccaggaaacgattagcctggttgtcgtcccgagcaatgtagatatcgcaaccaccgaggcattgtctatggcgcaagaagttgacccggaaggtgaccgtaccatcggtatcctgaccaaacctgacttggtggataagggtaccgaagataaagttgtggatgtcgtgcgtaatctggtgttccacctgaagaagggctacatgattgttaagtgtcgtggccagcaagagattcaggaccagttgagcctgagcgaggccctgcaacgtgagaaaatctttttcgagaaccacccgtattttcgcgacctgctggaagagggtaaggcgacggttccgtgcctggcagagaaattgacgtccgaattgattacccacatttgtaaatcgctgccgctgctggagaaccagatcaaagaaacccaccagcgcattaccgaagagctgcagaagtacggcgtcgacattccagaggatgaaaacgagaagatgtttttcctgattgataagatcaatgctttcaaccaagatattaccgcactgatgcaaggtgaagaaaccgtgggtgaagaggacatccgtctgttcacgcgtctgcgtcatgagtttcataaatggagcacgattatcgagaacaactttcaagagggccacaagattctgtctcgtaaaatccagaaatttgaaaaccagtatcgcggtcgtgagttgcctggtttcgtcaactaccgtaccttcgagactattgtgaagcaacagatcaaggcactggaagagccagcggttgatatgttgcatgacccggcagcgaacaaggcccgcaaagaggccgagctggctgcggcgaccgcggagcaataactcgag

SEQ ID NO: 4 - protein, 504 aa, Artificial sequence
  synthetic
MSGHHHHHHSGVVSEVDIAKADPAAASHPLLLNGDATVAQKNPGSVAENNLCSQYEEKVRPCIDLIDSLRALGVEQDLALPAIAVIGDQSSGKSSVLEALSGVALPRGSGIVTRCPLVLKLKKLVNEDKWRGKVSYQDYEIEISDASEVEKEINKAQNAIAGEGMGISHELITLEISSRDVPDLTLIDLPGITRVAVGNQPADIGYKIKTLIKKYIQRQETISLVVVPSNVDIATTEALSMAQEVDPEGDRTIGILTKPDLVDKGTEDKVVDVVRNLVFHLKKGYMIVKCRGQQEIQDQLSLSEALQREKIFFENHPYFRDLLEEGKATVPCLAEKLTSELITHICKSLPLLENQIKETHQRITEELQKYGVDIPEDENEKMFFLIDKINAFNQDITALMQGEETVGEEDIRLFTRLRHEFHKWSTIIENNFQEGHKILSRKIQKFENQYRGRELPGFVNYRTFETIVKQQIKALEEPAVDMLHDPAANKARKEAELAAATAEQ

SEQ ID NO: 5 - DNA, 207 bp, Artificial sequence
  synthetic
aattgtgagcggataacaattacgagcttcatgcacagtgaaatcatgaaaaatttatttgctttgtgagcggataacaattataatatgtggaattgtgagcgctcacaattccacaacggtttccctctagaaataattttgtttaacttttaggaggtaaaaaaaatgtaaccccaagggcgacaccccctaattagcccgggc

SEQ ID NO: 6 - DNA, 7 bp, Artificial sequence
  synthetic
aggaggt

SEQ ID NO: 7 - DNA, 48 bp, Artificial sequence
  synthetic
aaatcatgaaaaatttatttgctttgtgagcggataacaattataata

SEQ ID NO: 8 - DNA, 21 bp, Artificial sequence
  synthetic
aattgtgagcggataacaatt

SEQ ID NO: 9 - DNA, 30 bp, Artificial sequence
  synthetic
tgtggaattgtgagcgctcacaattccaca

SEQ ID NO: 10 - DNA, 151 bp, Artificial sequence
  synthetic
taatacgactcactataggggaattgtgagcggataacaattcccctctagaaataattttgtttaacttttaggaggtaaaaaaaatgtaaccccctagcataaccccttggggcctctaaacgggtcttgaggggttttttgcccctga

SEQ ID NO: 11 - DNA, 21 bp, Artificial sequence
  synthetic
taatacgactcactatagggg

SEQ ID NO: 12 - DNA, 21 bp, Artificial sequence
  synthetic
aattgtgagcggataacaatt

SEQ ID NO: 13 - DNA, 20 bp, Artificial sequence
  synthetic
aggaggtaaaaaaaatgtaa

SEQ ID NO: 14 - DNA, 48 bp, Artificial sequence
  synthetic
ctagcataaccccttggggcctctaaacgggtcttgaggggttttttg

SEQ ID NO: 15 - DNA, 2016 bp, Artificial sequence
  synthetic
atgagcggtcatcaccatcatcatcacagcggcgtagtatcggaagtagacattgcaaaagccgatccagctgcggccagccacccactgctgctgaatggtgacgcaaccgtggcgcaaaagaatccgggctccgttgcggagaataacctgtgtagccagtatgaagagaaggttcgtccgtgtattgacctgatcgactccctgcgtgcgctgggtgtggagcaggacttggcactgccggctattgcggtcatcggtgatcaatctagcggtaagagcagcgtcctggaggcgctgagcggtgttgcgctgccgcgtggtagcggcattgtcacccgttgccctctggtcttgaaactgaagaaactggtcaatgaagataaatggcgtggcaaagtgagctaccaggattacgaaatcgagatcagcgacgcatccgaggtggaaaaagaaatcaataaagctcagaatgcgatcgcgggtgagggtatgggcattagccacgagctgattaccctggaaatttcgagccgcgatgtcccggacctgaccctgatcgatctgccgggtattacgcgcgtcgcagttggcaaccagccagcggacattggctacaagatcaaaacgctgattaagaagtatatccaacgtcaagaaaccattagcttggttgtggtgccgagcaacgttgacattgcgaccacggaggccctgagcatggcacaagaggttgatccggagggtgaccgcaccatcggcattctgacgaagccggacctggtggataaaggtacggaggacaaagtggtggacgtggttcgcaatctggtttttcacttgaagaaaggttacatgattgttaaatgccgtggtcaacaggagattcaagatcagctgagcctgagcgaggccctgcagcgcgagaaaatcttcttcgagaaccacccgtatttccgtgatctgctggaggaaggtaaggccacggtgccgtgcctggcggagaaactgaccagcgagctgatcacccatatctgcaaatccctgccgctgctggagaaccagatcaaggaaacgcatcagcgtatcaccgaagagttgcagaaatatggtgttgatatcccggaggatgagaacgagaagatgttctttctgattgataagatcaacgctttcaaccaagacattactgcgctgatgcaaggcgaagaaacggttggtgaggaagatattcgtctgttcacccgtctgcgccacgagtttcataagtggagcacgattatcgagaataactttcaagaaggccacaaaatcctgagccgtaagatccaaaagtttgagaaccaataccgtggtcgtgagctgccgggttttgtcaattaccgtacttttgaaaccattgtgaaacagcagattaaggcattggaggaaccggctgtggatatgctgcacaccgtcaccgacatggttcgtctggcctttaccgatgtttccatcaagaacttcgaagagttcttcaatctgcatcgtacggcgaagagcaagattgaggacattcgcgccgaacaggaacgcgagggtgagaagctgatccgcctgcactttcagatggagcagattgtgtactgtcaagatcaagtttaccgtggtgcgctgcagaaagtccgtgaaaaagaactggaagaggaaaagaagaaaaagagctgggatttcggcgcgtttcagagcagctcggcaaccgactcctctatggaagagatttttcaacacttgatggcctatcaccaggaagcttctaagcgtatctccagccacatcccgctgatcatccaattcttcatgttgcagacctatggccagcaactgcaaaaagcgatgctgcagctgctgcaagacaaagacacgtacagctggttgctgaaagaacgcagcgacaccagcgacaaacgcaaatttctgaaggaacgtttggcccgcttgacccaagcacgtcgtcgcctggcacagttcccgggc

SEQ ID NO: 16 - DNA, 1512 bp, Artificial sequence
  synthetic
atgagcggtcaccatcatcaccaccatagcggcgtagtaagcgaagtcgatattgcaaaagcggatccggctgcagcgtcgcacccgctgctgctgaatggtgatgcgacggtggcccagaagaatccgggtagcgttgcggagaacaatttgtgtagccagtacgaagagaaagttcgtccgtgcatcgacctgatcgacagcctgcgtgcgctgggcgttgaacaggacctggcactgccggcgatcgcagtcatcggtgatcaatccagcggcaaatcttccgtcctggaagctctgagcggtgtggcgctgccgcgtggcagcggcattgtcacgcgctgcccgttggtgctgaaactgaaaaagctggtgaatgaggacaaatggcgcggtaaggtgtcctaccaggactatgagattgaaattagcgacgctagcgaagttgaaaaagagatcaataaagcgcaaaacgcgattgccggtgagggcatgggtatcagccacgaactgatcacgctggagatcagcagccgcgatgtcccggacctgaccctgattgacctgccgggtatcactcgtgttgcggttggcaatcaaccggccgatattggctacaagattaagaccctgatcaaaaagtatatccagcgccaggaaacgattagcctggttgtcgtcccgagcaatgtagatatcgcaaccaccgaggcattgtctatggcgcaagaagttgacccggaaggtgaccgtaccatcggtatcctgaccaaacctgacttggtggataagggtaccgaagataaagttgtggatgtcgtgcgtaatctggtgttccacctgaagaagggctacatgattgttaagtgtcgtggccagcaagagattcaggaccagttgagcctgagcgaggccctgcaacgtgagaaaatctttttcgagaaccacccgtattttcgcgacctgctggaagagggtaaggcgacggttccgtgcctggcagagaaattgacgtccgaattgattacccacatttgtaaatcgctgccgctgctggagaaccagatcaaagaaacccaccagcgcattaccgaagagctgcagaagtacggcgtcgacattccagaggatgaaaacgagaagatgtttttcctgattgataagatcaatgctttcaaccaagatattaccgcactgatgcaaggtgaagaaaccgtgggtgaagaggacatccgtctgttcacgcgtctgcgtcatgagtttcataaatggagcacgattatcgagaacaactttcaagagggccacaagattctgtctcgtaaaatccagaaatttgaaaaccagtatcgcggtcgtgagttgcctggtttcgtcaactaccgtaccttcgagactattgtgaagcaacagatcaaggcactggaagagccagcggttgatatgttgcatgacccggcagcgaacaaggcccgcaaagaggccgagctggctgcggcgaccgcggagcaa

(19) United States
(12) Patent Application Publication
(10) Pub. No.: US 2013/0344565 A1
(43) Pub. Date: Dec. 26, 2013

(54) Optimization of Expression and Purification of Recombinant Human MxA Protein in E. Coli

(71) Applicant: RAPID PATHOGEN SCREENING, INC., Sarasota, FL (US)

(72) Inventors: Robert W. VanDine, Montoursville, PA (US); Robert P. Sambursky, Bradenton, FL (US); Uma Mahesh Babu, Bradenton, FL (US); Kyosuke Nagata, Tsukuba (JP)

(73) Assignee: RAPID PATHOGEN SCREENING, INC., Sarasota, FL (US)

(21) Appl. No.: 13/788,716

(22) Filed: Mar. 7, 2013

Related U.S. Application Data
(60) Provisional application No. 61/662,656, filed on Jun. 21, 2012.

(51) Int. Cl. C12N 9/14

(52) U.S. Cl. CPC C12N 9/14
USPC 435/195; 435/320.1

(57) ABSTRACT

Full length MxA constructs and truncated MxA constructs produce human MxA protein in E. coli. The full length MxA and truncated MxA constructs are preferably E. coli codon-optimized to optimize the amount of protein made using the constructs. T5 or T7 promoters can each be used in combination with either the full length MxA or the truncated MxA constructs. In one preferred embodiment, the MxA protein produced by the full length MxA or truncated MxA constructs is used in a control prep or external control. In other preferred embodiments, the MxA protein is used as a therapeutic.

### REFERENCE TO RELATED APPLICATIONS

This application claims one or more inventions which were disclosed in Provisional Application No. 61/662,656, filed Jun. 21, 2012, entitled “OPTIMIZATION OF EXPRESSION AND PURIFICATION OF RECOMBINANT HUMAN MxA PROTEIN IN E. COLI”. The benefit under 35 USC §119(e) of the United States provisional application is hereby claimed, and the aforementioned application is hereby incorporated herein by reference.

### BACKGROUND OF THE INVENTION

1. Field of the Invention

The invention pertains to the field of the expression of proteins. More particularly, the invention pertains to the expression of truncated and full length recombinant MxA protein in E. coli.

2. Description of Related Art

U.S. Pat. No. 6,180,102, issued Jan. 30, 2001, entitled “Monoclonal antibody to human MxA protein”, and incorporated herein by reference, cloned a full-length MxA-encoding cDNA into a plasmid and introduced it into E. coli cells using a vector with a T7 promoter.

### SUMMARY OF THE INVENTION

Full length MxA constructs and truncated MxA constructs produce human MxA protein in E. coli. The full length MxA and truncated MxA constructs are preferably codon-optimized to optimize the amount of protein made using the constructs in E. coli. T5 or T7 promoters can each be used in combination with either the full length MxA or the truncated MxA constructs. In one preferred embodiment, the MxA protein produced by the full length MxA or truncated MxA constructs is used in a control prep, as the assay standard, or as or an external control. In other preferred embodiments, the MxA protein is used as a therapeutic.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows a predicted tertiary structure for a full length MxA codon optimized clone.

FIG. 2 shows a predicted tertiary structure for a truncated MxA codon optimized clone.

FIG. 3 shows transcriptional control of a T7 gene in lambda-DE3 lysogens in an example of a T7 cloning vector.

FIG. 4 shows an SDS-PAGE analysis of control preps.

FIG. 5 shows a scan of an ELISA plate.

FIG. 6 shows a standard curve of His tag protein levels per well versus an Ab 412 nm ELISA signal.

FIG. 7 shows a Western analysis of MxA preps.

FIG. 8 shows an SDS-PAGE analysis of MxA ΔC expression in BL21 (DE3) under various conditions of induction.

FIG. 9 shows an SDS PAGE comparison of calculated 10 μg of refolded ΔC-MxA protein produced from T7 (lane 1) and T5 (lane 2) promoter driven expression constructs.

FIG. 10 shows a Western and SDS PAGE analysis of the refolded ΔC-MxA protein produced using the T5 expression construct in BL21 (DE3).

FIG. 11 shows quantification of the ΔC-MxA protein by ELISA.

FIG. 12 shows a Bradford assay determination of protein concentration.

### DETAILED DESCRIPTION OF THE INVENTION

Full length MxA constructs and truncated MxA constructs produce human MxA protein in E. coli. The full length MxA and truncated MxA constructs are preferably codon-optimized to optimize the amount of protein made using the constructs. T5 or T7 promoters can each be used in combination with either the full length MxA or the truncated MxA constructs. In one preferred embodiment, the MxA protein produced by the full length MxA or truncated MxA constructs is used in a control prep, as the assay standard, or as an external control. In other preferred embodiments, the MxA protein produced by the full length MxA or truncated MxA constructs is used as a therapeutic.

The truncated MxA constructs disclosed herein produce MxA protein that is more stable than the full length MxA protein. The T5 and T7 promoters can each be used in combination with either the full length MxA or the truncated MxA constructs described herein. Embodiments disclosed herein preferably use a T5 promoter, which is equally robust as a T7 promoter.

The full length MxA protein tends to fold on itself and has stability issues. The C-terminal truncated construct is much more stable and does not fold on itself. In addition to folding on itself, the full length MxA protein gives variable quantification results. The truncated MxA protein does not fold and maintains its activity for several days at refrigerated temperatures. The biological activity with the preservation of the GTPase binding areas in both the full length and the shorter C-terminal truncated moieties should be the same.

In addition, since the C-terminal truncated moiety is about 30% smaller (molecular weight is about 55,000 Daltons), it should be more “tolerable” in injections than the 30% larger full length moiety. So, the C-terminal truncated moiety creates less immunogenic problems than the full length moiety.

In preferred embodiments, the full length and truncated MxA constructs include a T5 promoter. In other embodiments, a T7 promoter is used. In still other embodiments, other promoters, including, but not limited to, lac, lacUV5, tac (hybrid) (differs from the trc promoter by 1 bp), trc (hybrid) (differs from the tac promoter by 1 bp), trp, araBAD, phoA, recA, proU, cst-1, tetA, cadA, nar, pL, cspA, SP6, T3-lac operator, and T4 gene 32, may be used.

Laboratory-based immunoassays based on chemiluminescence, immunofluorescence, surface plasmon resonance, electro-chemical or ELISA testing or point of care tests for human MxA protein require an external control and standard. For example, human MxA protein is used as a control component of a screen for the differentiation and detection of viral versus non-viral (for example, bacterial) infection in humans. In some embodiments, the full length or truncated MxA constructs described herein supply human MxA as that external control. The concentration of MxA protein is calibrated to the titration of this standard for example in ELISA testing. This external control, also described as a “control prep” herein, is utilized by hospitals and research laboratories to ensure that their inventory of MxA reagents remains fresh and usable.

The truncated MxA proteins described herein are more stable than the full length proteins. Therefore, they are preferred for use as the external standard or control prep because they have a longer shelf life. The prior art uses only a full length chain for any use of MxA, including the use as a control prep.

In preferred embodiments, both the full length and truncated moieties are preferably produced with a Histidine tail (six His), which allows for easy purification on a Nickel (or another metal) column. In some embodiments with the six His tail, there is a post-translation step that removes the six His tail.

Since the protein sequence is not altered, all homologies between the human MxA and animal MxA protein is maintained. This means that the MxA proteins being produced can be used in veterinary applications.

Very large quantities of MxA protein (truncated or full length) can be produced using the constructs and methods described herein.

In addition, because the truncated and full length constructs described herein are preferably codon-optimized, they result in production of higher amounts of usable protein.

Producing human MxA for External Control Prep

The human MxA protein is produced recombinantly in E. coli for use as a control component of a screen for the detection of viral versus bacterial infection in humans. Two constructs, a full length MxA cDNA clone (SEQ. ID. NO. 1) and a C-terminal truncated form of the MxA cDNA clone (SEQ. ID. NO. 3), are used to produce the recombinant MxA protein (SEQ. ID. NOS. 2 and 4, respectively). The protein (SEQ. ID. NO. 4) produced from the truncated clone appears to have higher stability than the full length form (SEQ. ID. NO. 2) and is the preferred form of MxA utilized in the screen. SEQ. ID. NO. 15 shows an alternative full-length MxA cDNA clone, without the nine non-coding base pairs in SEQ. ID. NO. 1. SEQ. ID. NO. 16 shows an alternative C-terminal truncated form of the MxA cDNA clone, without the nine non-coding base pairs in SEQ. ID. NO. 3.

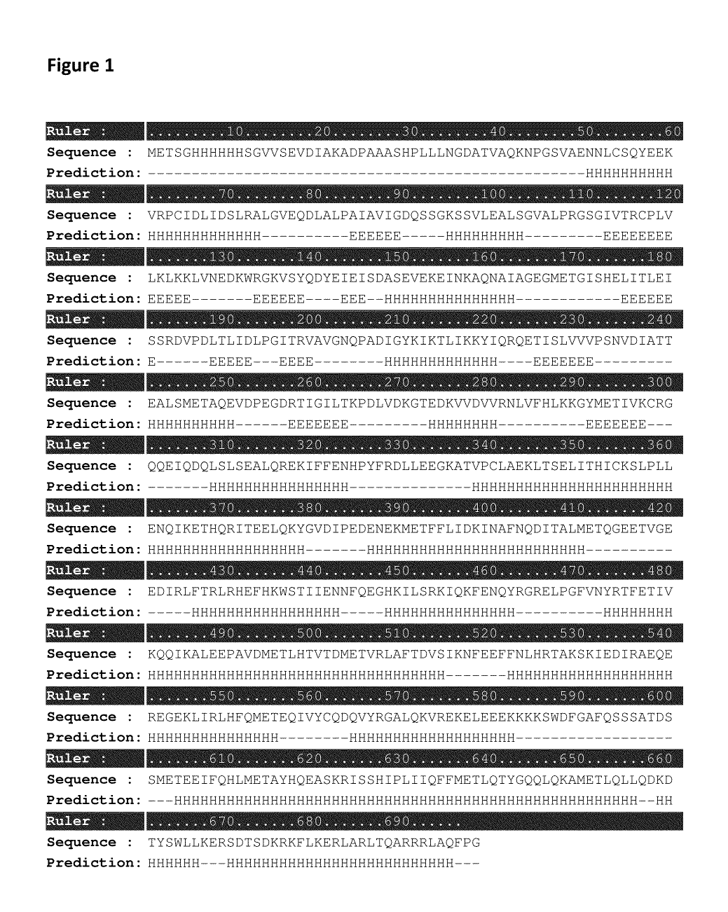

SEQ. ID. NO. 1 shows a DNA sequence of a codon-optimized full-length MxA protein. SEQ. ID. NO. 1 includes 2025 base pairs. SEQ. ID. NO. 15 also shows a DNA sequence of a codon-optimized full length MxA protein, with 2016 base pairs. These sequences are the same, except that SEQ. ID. NO. 1 includes nine non-coding base pairs at the very end of the DNA sequence of SEQ. ID. NO. 1. SEQ. ID. NO. 2 shows the MxA protein for SEQ. ID. NO. 1 and SEQ. ID. NO. 15. The predicted isolectric point (pI) for SEQ. ID. NO. 2 is 5.30 and its predicted molecular weight is 79638.35. FIG. 1 shows the predicted tertiary structure for SEQ. ID. NO. 2. Note that methionine is listed as “MET” in this figure, instead of using its one letter code.

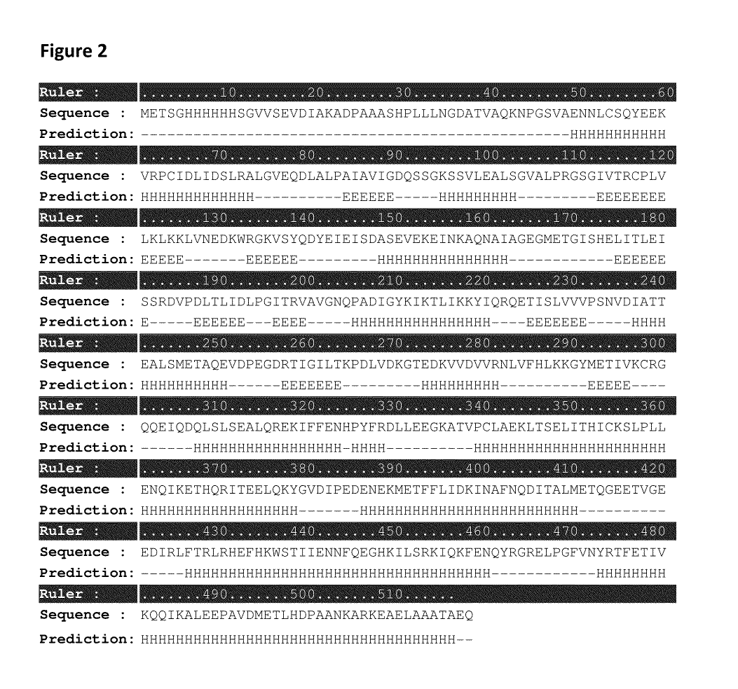

SEQ. ID. NO. 3 shows a DNA sequence of a codon-optimized truncated MxA protein. SEQ. ID. NO. 3 includes 1521 base pairs. SEQ. ID. NO. 4 shows the MxA protein for SEQ. ID. NO. 3. The predicted isolectric point (pI) for SEQ. ID. NO. 4 is 5.07 and its predicted molecular weight is 57894.62. FIG. 2 shows the predicted tertiary structure for SEQ. ID. NO. 4. Note that methionine is listed as “MET” in this figure, instead of using its one letter code. SEQ. ID. NO. 16 shows another DNA sequence of a codon-optimized truncated MxA protein. SEQ. ID. NO. 16 includes 1500 base pairs. SEQ. ID. NO. 3 and SEQ. ID. NO. 16 are the same, except that SEQ. ID. NO. 3 includes nine non-coding base pairs at the very end of the DNA sequence of SEQ. ID. NO. 3.

The full length (SEQ. ID. NO. 2 and SEQ. ID. NO. 15) and truncated (SEQ. ID. NO. 4 and SEQ. ID. NO. 16) codon-optimized clones are preferably expressed in the pJExpress 401 vector (SEQ. ID. NO. 5), which is kanamycin-resistant. The pJExpress 401 vector includes a ribosome binding site (RBS) (SEQ. ID. NO. 6), a T5 promoter (SEQ. ID. NO. 7), a Lac operator sequence (SEQ. ID. NOS. 8 and 9) and an open reading frame (ATG . . . TAA). The lac operator sequence is responsible for the protein expression induction with the inclusion of IPTG (Isopropyl-β-D-thiogalacto pyranoside). Other T5 cloning cassettes known in the art could be alternative used.

While the full length and truncated forms of the MxA constructs are preferably cloned into the pJExpress 401 vector with T5, in other embodiments, the constructs are cloned into an expression vector utilizing a T7 promoter.

SEQ. ID. NO. 10 shows a cloning cassette with a T7 promoter (SEQ. ID. NO. 11), a lacO1 (SEQ. ID. NO. 12), an RBS (consensus E. coli RBS shown), a spacer, an open reading frame, (ATG . . . TAA), and aT7 terminator (SEQ. ID. NO. 14). SEQ. ID. NO. 13 shows a sequence including the RBS, spacer, and open reading frame.

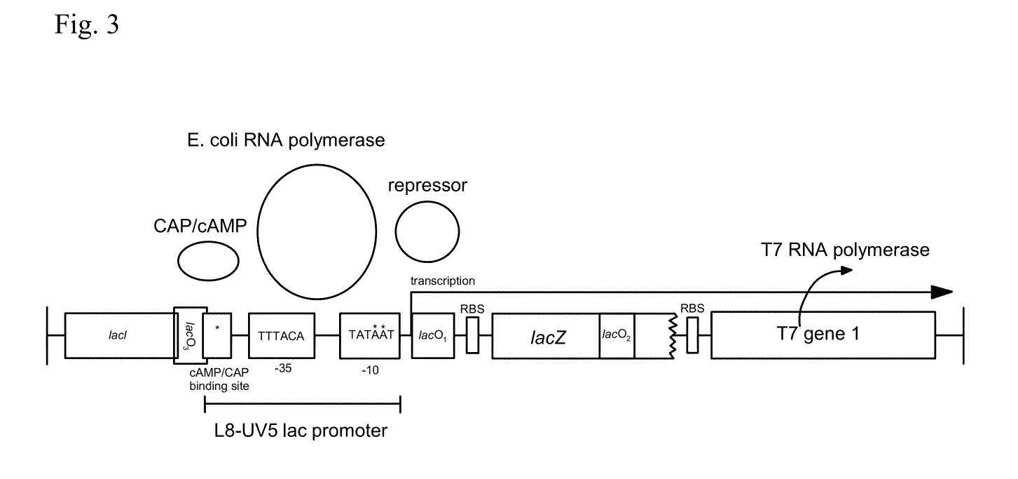

FIG. 3 shows an example of a pET E. coli T7 expression vector (Studier and Moffatt, “Use of bacteriophage T7 RNA polymerase to direct selective high-level expression of cloned genes”, J Mol Biol. 1986 May 5; 189(1):113-30; Rosenberg, A. H., Lade, B. N., Chui, D., Lin, S., Dunn, J. J., and Studier, F. W. (1987) Gene 56, pp. 125-135; and Studier, F. W., Rosenberg, A. H., Dunn, J. J., and Dubendorff, J. W. (1990) Meth. Enzymol. 185, pp. 60-89, all herein incorporated by reference) from Novagen (Merck Millipore).

The pET plasmids contain an expression cassette in which the gene of interest is inserted behind a strong T7 promoter. In the absence of the T7 polymerase, the T7 promoter is off. For expression, the pET plasmids are transformed into bacteria strains that typically contain a single copy of the T7 polymerase on the chromosome in a lambda lysogen (for example, the DE3 lysogen). In the example shown in FIG. 3, the Lac-UV5 lac promoter controls the T7 polymerase. When cells are grown in media without lactose, the lac repressor (lad) binds to the lac operator and prevents transcription from the lac promoter. When lactose or IPTG (a lactose analog) is added to the media, lactose (or IPTG) binds to the repressor and reduces its affinity for the operator, permitting transcription from the promoter. In the absence of glucose, CAP/cAMP levels are sufficiently high to form a CAP/cAMP complex that binds upstream of the promoter to best stimulate transcription. When glucose is added, CAP/cAMP is not formed, and transcription decreases. Other pET T7 expression vectors or other T7 cloning cassettes known in the art could be alternatively used.

In other embodiments, other protein expression vectors and systems could be used with promoters other than T5 or T7.

In some embodiments, both the full length and truncated MxA clones are cloned into an expression vector utilizing a T7 promoter to drive expression and carry N-terminal 6×His tags for affinity purification. Additional expression constructs have been codon-optimized for expression in E. coli and utilize either the T5 promoter or the T7 promoter to drive expression. In a preferred embodiment, the T5 promoter is used to drive expression of a codon-optimized truncated MxA construct.

Preparations of the human MxA were generated using various protocols. A goal is to generate a control prep using a provided protocol, and then optimize expression and purification of the truncated form from E. coli.

A crude control preparation of the full-length MxA protein was generated in E. coli BL21 (DE3) cells using the protocol outlined in U.S. Pat. No. 6,180,102, issued Jan. 31, 2001, herein incorporated by reference (see, for example column 7, lines 28-62, and FIG. 1 of U.S. Pat. No. 6,180,102).

The preparations were created using both a T7-promoter expression construct and the codon-optimized T5 promoter construct. These preps are referred to as “control preps” herein. Since the protein is not highly purified using this method, purity was estimated using ELISA detection of the 6×His tag.

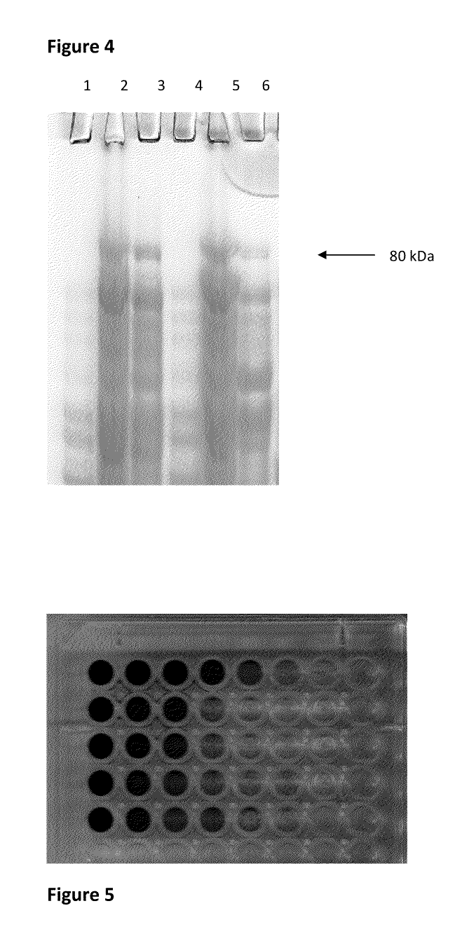

FIG. 4 shows SDS-PAGE analysis of control preps. Proteins were prepared using the control prep method from both untransformed and transformed BL21. The proteins were separated on a 4-12% acrylamide gradient gel and stained with Coomassie blue protein stain. Lane 1 shows the un-induced BL21 T5 promoter. Lane 2 shows a T5-construct transformed BL21. Lane 3 shows a T5-construct transformed BL21. Lane 4 shows an uninduced BL21 T7 promoter. Lane 5 shows a T7-construct transformed BL21. Lane 6 shows a T7-construct transformed BL21. The SDS-PAGE analysis indicated that the preps were not very pure but rather enriched in MxA protein.

Since the SDS PAGE gels of these preps indicated that the protein was not >10% pure, ELISA using anti-his tag antibody conjugated with HRP was used to estimate the levels of MxA protein in each of the preps. In addition, a Western blot of the preparations was also run.

ELISA Assay

ELISA plates were coated overnight at 4° C. with serial dilutions of the MxA preparation and a control His tagged protein of known concentration. The plates were washed and blocked with PBS+2% BSA for 2 hours at room temperature. The plates were then incubated with mouse anti-His antibody (GE 1:3000 dilution) for 2 hours at room temperature, after which the plates were washed 3×15 mins with PBS+0.05% Tween. The plates were then incubated with goat-anti-mouse HRP conjugate (Lampire Biological Labs 1:3000 dilution) for 1 hour at room temperature, after which the plates were washed 3×15 mins with PBS+Tween 0.05%. The signal was detected using ABTS peroxidase substrate (KPL) and the plates read at 412 nm wavelength.

FIG. 5 shows an ELISA determination of the MxA content of the control prep. Serial dilutions of the protein preps were prepared and each well loaded with a determined volume load. A standard curve was prepared using a known concentration of His-tagged protein, and from this curve, the protein concentration of the MxA preps was calculated.

Lane A (top lane) shows a control protein serial dilution. In this lane, the wells include: well 1: 2 μg control protein; well 2: 1 μg; well 3: 500 ng; well 4: 250 ng; well 5: 100 ng; well 6: 40 ng, well 7: 20 ng; well 8: 10 ng.

Lane B shows serial dilutions of MxA prep A (Amp-T7 plasmid control 1). In this lane, the wells include: well 1: 10 μL; well 2: 2.5 μL; well 3: 1 μL; well 4: 0.1 μL; well 5: 0.05 μL; well 6: 0.025 μL; well 7: 0.01 μL; well 8: 0.005 μL.

Lane C shows serial dilutions of MxA prep B (Amp-T7 plasmid control 2). In this lane, the wells include: well 1: 10 μL; well 2: 2.5 μL; well 3: 1 μL; well 4: 0.1 μL; well 5: 0.05 μL; well 6: 0.025 μL; well 7: 0.01 μL; well 8: 0.005 μL.

Lane D shows serial dilutions of MxA prep C (Kan-T5 plasmid control 3). In this lane, the wells include: well 1: 10 μL; well 2: 2.5 μL; well 3: 1 μL; well 4: 0.1 μL; well 5: 0.05 μL; well 6: 0.025 μL; well 7: 0.01 μL; well 8: 0.005 μL.

Lane E shows serial dilutions of MxA prep D (Kan-T5 plasmid control 4). In this lane, the wells include: well 1: 10 μL; well 2: 2.5 μL; well 3: 1 μL; well 4: 0.1 μL; well 5: 0.05 μL; well 6: 0.025 μL; well 7: 0.01 μL; well 8: 0.005 μL.

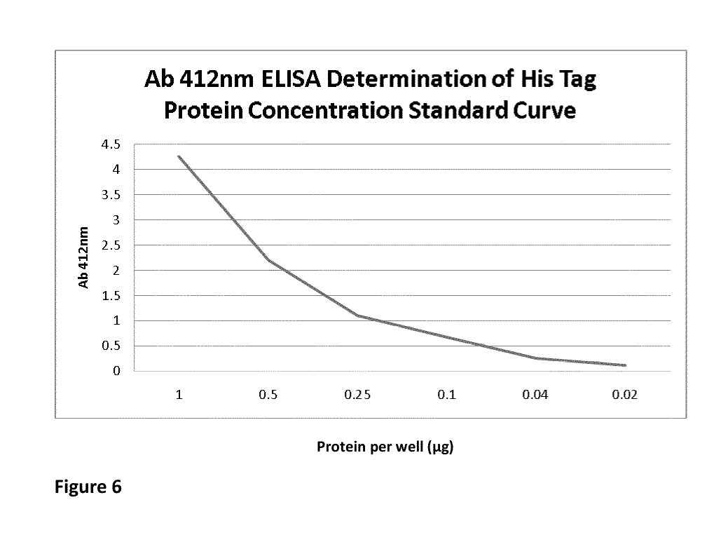

FIG. 6 shows a standard curve of His Tag protein levels per well versus an Ab412 nm ELISA signal. Based on this data, the His-tag MxA concentration of prep A (T7 plasmid control 1) was 22 ng/0.1 μL=0.22 μg/mL. The His-tag MxA concentration of prep B (T7 plasmid control 2) was 20 ng/0.075 μL=0.26 μg/mL. The His-tag MxA concentration of prep C (T5 plasmid control 3) was 28 ng/0.05 μL=0.56 μg/mL. The His-tag MxA concentration of prep D (T5 plasmid control 4) was 20 ng/0.025 μL=0.8 μg/mL.

Western Blot

Western analysis was based on a similar protocol: the proteins were separated by SDS PAGE before transfer to a nylon membrane. The membrane was blocked overnight in PBS+2% BSA, washed 3×15 mins in PBS+Tween 0.05% before incubation with mouse anti-His antibody (GE, 1:3000 dilution) for 2 hours at room temperature. The membrane was then washed 3×15 mins with PBS+Tween 0.05% before incubation with the secondary goat-anti-mouse HRP conjugate for 2 hours at room temperature. The membrane was then washed 3×15 mins with PBS+Tween 0.05% and transferred to ABTS peroxidase substrate for detection.

Samples of a 104, volume from each of the 4 preps (A, B, C and D) were separated by SDS-PAGE (4-12% acrylamide gradient gel) and transferred to the nylon membrane. The His-tagged protein was detected on the blot using mouse anti-His antibody, and goat-anti-mouse-HRP conjugate.

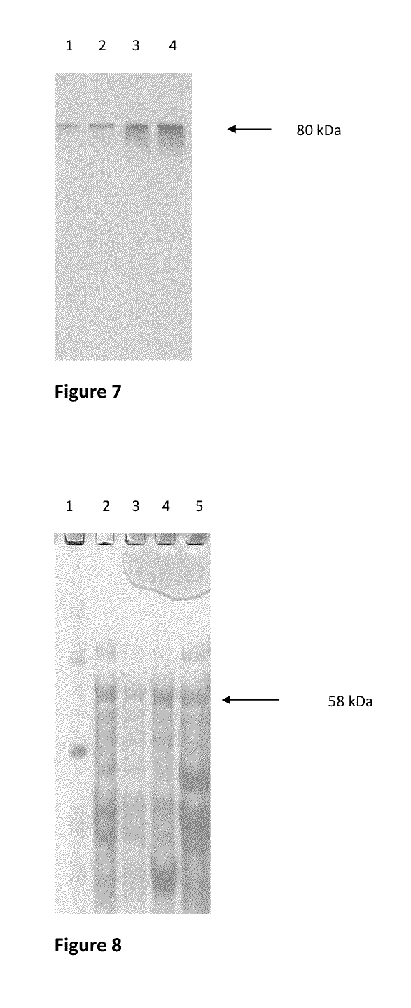

FIG. 7 shows the results of the Western analysis of the MxA preps. Lane 1 was the T7-prep A (control plasmid 1), lane 2 was the T7 prep B (control plasmid 2), lane 3 was the T5 prep C (control plasmid 3) and lane 4 was the T5 prep D (control plasmid 4). The Western analysis identified a single protein product in each of the 4 control preps. This protein band correlates to an 80 kDa protein, which roughly corresponds to the size of the human MxA protein.

Expression and Purification C-Terminal Truncated MxA from Expression Vectors Utilizing the T7 and T5 Promoters

Expression constructs encoding His-tagged human MxA C-terminal truncated protein were transformed into BL21 (DE3) E. coli. The bacteria were grown at 28° C. in terrific broth (1.2% tryptone, 2.4% yeast extract, 0.4% glycerol, buffered to pH 7.2 with phosphate buffer) medium containing either 50 μg/mL ampicillin (T7 promoter vector) or 25 μg/mL kanamycin (T5 promoter vector). When the exponentially growing cultures reached an OD₆₀₀ₙₘ of 0.4, they were induced by the addition of 0.1 mM IPTG for 6 hours. The cells were harvested and washed with ice cold PBS and resuspended in ice cold buffer A containing 50 mM Tris-HCl (pH 8.0), 500 mM NaCl, 5 mM MgCl₂, 10 mM 2-mercaptoethanol, 10% glycerol, 0.1% Nonidet P-40, 2 mM imidazole and protease inhibitors. Sonication on an ice bath was performed to lyse the cells and the cell debris removed by centrifugation (10,000×g for 30 mins 4° C.). The clarified supernatant was loaded onto a Ni-resin column (Qiagen) and the column washed with 10 column volumes of buffer A and then 10 column volumes of buffer B containing 20 mM Tris-HCl (pH 8.0), 100 mM KCl, 5 mM MgCl₂, 20% glycerol, 0.1% Nonidet NP-40, 20 mM imidazole. The protein was then eluted in buffer B containing 200 mM imidazole.

In order to determine the MxA concentrations per prep, an ELISA analysis using anti-His antibody was used.

Optimization of C-Terminal Truncated MxA Expression in E. coli

Codon optimization is a process by which an organism is examined to see what codons are used rarely and what codons are used more frequently for particular proteins in that organism. The codons that are used rarely reduce the tRNA, making them less efficient at making proteins. Codons that are used more frequently make more tRNA, and consequently grow proteins faster. Computer programs figure out what codons to change to optimize the protein produced, and a DNA synthesizer is used to change the codons.

Several conditions of protein expression of the C-terminal truncated form of the MxA protein have been analyzed.

BL21 (DE3) cells were transformed with the T5-promoter-ΔC MxA constructs and cultures were grown and induced under the specified conditions. The bacteria were grown at 28° C. in terrific broth (1.2% tryptone, 2.4% yeast extract, 0.4% glycerol, buffered to pH 7.2 with phosphate buffer) medium containing either 50 μg/ml ampicillin (T7 promoter vector) or 25 μg/ml kanamycin (T5 promoter vector). When the exponentially growing cultures reached an OD600 nM of 0.4, they were induced by the addition of IPTG (Isopropyl β-D-Thiogalacto pyranoside).

Cell lysates were generated by sonication and loaded onto Ni-spin columns for His-tag protein enrichment. This step is intended as an expression analysis tool rather than protein purification.

FIG. 8 shows an SDS-PAGE analysis of MxA ΔC expression in BL21 (DE3) cells under various conditions of induction. Lane 1 is uninduced MxA ΔC expression, lane 2 is 28° C., induction with 0.1 mM IPTG, lane 3 is 28° C., induction with 0.01 mM IPTG, lane 4 is 30° C., induction with 0.1 mM IPTG and lane 5 is 30° C. pre induction benzyl alcohol treatment, induction with 0.01 mM IPTG.

The initial data indicates that there is good induction of the MxA ΔC-terminal truncated protein at both 28° C. and 30° C. The use of 0.1 mM IPTG appears more effective than 0.01 mM at both temperatures. Furthermore, the pre-induction treatment with Benzyl alcohol (a chaperone activator) appears to increase levels of soluble MxA expression.

Optimization of ΔC-MxA Protein Recovery and Refolding from Inclusion Bodies

In order to maximize the production of soluble ΔC-MxA protein from inclusion bodies, the process began with the optimization of the harvesting and processing of the inclusion bodies from BL21 (DE3) cells. A stringent washing procedure was employed for the inclusion bodies, which removed as many contaminating proteins as possible early on before complete solubilization. Re-precipitation of the solubilized protein by diluting out the Guanidine HCl effectively removed some of the small protein contaminants of the inclusion body prep; it also enabled the use of βME and EDTA for the initial inclusion body disruption and solubilization but allowed them to be removed prior to affinity purification, since both of these reagents are incompatible with the use of metal affinity resins.

The complete procedure is summarized below:

The frozen cell pellets were re-suspended in 50 mM Tris-HCl, 200 mM NaCl pH 8.0. The cells were lysed by sonication (total of 10 mins pulse treatment 2 seconds on, 2 seconds off) and the suspension centrifuged at 5,000×g for 30 mins at 4° C. The supernatant was removed and discarded and the pellet washed by re-suspension in 50 mM Tris HCl, 500 mM NaCl, 2% Triton X-100, 2 mM βME, 2M urea pH 8.0 and centrifugation at 17,000×g for 20 mins at 4° C. This process was repeated 5 times, followed by a wash in 50 mM Tris HCl, pH 8.0 and centrifugation at 17,000×g for 20 mins at 4° C. The pellet was then re-suspended in 50 mM Tris-HCl, 50 mM NaCl, 1 mM EDTA, 100 mM βME, 7M Guanidine Hydrochloride (Gdn HCl) pH 8.0. The suspension was mixed overnight at 4° C. to completely solubilize the inclusion body protein. The following morning, the suspension was centrifuged at 17,000×g for 20 mins at 4° C. and insoluble material discarded.

The solubilized protein was re-precipitated by the addition of 6 volumes 50 mM Tris-HCl, 50 mM NaCl, 1 mM EDTA, 100 mM βME pH 8.0. The precipitated protein was harvested by centrifugation at 30,000×g for 20 mins at 4° C. and resuspended in 50 mM Tris-HCl, 250 mM NaCl, 10 mM imidazole, 6M Gdn HCl pH 8.0. The preparation was mixed with Talon® resin for 2 hrs at 4° C. in order to completely capture the His-tag ΔC-MxA protein. The resin was harvested by centrifugation at 500×g for 10 mins at 4° C., and then washed for 45 mins at 4° C. in 40 column volumes of 50 mM Tris-HCl, 250 mM NaCl, 10 mM imidazole, 6M Gdn HCl pH 8.0. The resin was harvested by centrifugation at 500×g for 10 mins at 4° C., and then aliquoted for refolding testing and optimization.

A buffer matrix was prepared using the base refolding buffers listed below and supplemented immediately before use with βME, GSH, GSSG, PEG 30,000, and EDTA as described below, and the resin (representing ˜100 ug of ΔC-MxA) was added into each of the refolding buffers. After 24 hrs at 4° C. with gentle shaking, the resin was harvested by centrifugation and the protein eluted in 50 mM Tris-HCl, 250 mM NaCl, 200 mM imidazole, 10% glycerol. Soluble protein yields were then determined using Ab280 nm to measure protein concentration. The results are summarized as a percentage of the total ΔC-MxA protein added on the Talon resin to each sample.

Final working concentrations:
Buffer 1. 50 mM Tris, 20 mM NaCl, 0.8 mM KCl pH 8.2
Buffer 2. 40 mM L-arginine, 50 mM Tris, 20 mM NaCl, 0.8 mM KCl pH 8.2
Buffer 3. 80 mM L-arginine, 50 mM Tris, 20 mM NaCl, 0.8 mM KCl pH 8.2
Buffer 4. 500 mM guanidine, 50 mM Tris, 20 mM NaCl, 0.8 mM KCl, pH 8.2
Buffer 5. 500 mM guanidine, 400 mM L-arginine, 50 mM Tris, 20 mM NaCl, 0.8 mM KCl pH 8.2
Buffer 6. 500 mM guanidine, 800 mM L-arginine, 50 mM Tris, 20 mM NaCl, 0.8 mM KCl, pH 8.2
Buffer 7. 1M guanidine, 50 mM Tris, 20 mM NaCl, 0.8 mM KCl, pH 8.2
Buffer 8. 1M guanidine, 40 mM L-arginine, 50 mM Tris, 20 mM NaCl, 0.8 mM KCl, pH 8.2
Buffer 9. 1M guanidine, 80 mM L-arginine, 50 mM Tris, 20 mM NaCl, 0.8 mM KCl, pH 8.2
Buffer 10. 20 mM Tris, 100 mM KCl, 5 mM MgCl₂, 20% glycerol, 0.1% Nonidet P-40, pH 8.0
Buffer 11. 100 mM Tris, 400 mM L-arginine pH 8.0
Buffer 12. 800 mM L-arginine, 50 mM Tris, 20 mM NaCl, 0.8 mM KCl

MxA protein added on the Talon resin to each sample (assuming that 100 μg of ΔC-MxA bound to 80 μL of Talon suspension is added per refolding reaction; binding capacity of Talon resin for ΔC-MxA is 5 mg protein per mL settled resin, the suspension is used as a 50:50 resin to buffer suspension). Table 1 shows percentage soluble protein recovery for each buffer.

TABLE 1

| | |
|---|---|
| Buffer 1 plus 1 mM EDTA, 2 mM GSH, 0.2 mM | Buffer 1 plus 1 mM EDTA, 2 mM GSH, 0.2 mM |
| GSSG | GSSG, 1 mM PEG (15%) |
| (27%) | |
| Buffer 2 plus 1 mM EDTA, 2 mM GSH, 0.4 mM | Buffer 2 plus 1 mM EDTA, 2 mM GSH, 0.4 mM |
| GSSG | GSSG, 1 mM PEG (14%) |
| (15%) | |
| Buffer 3 plus 1 mM EDTA, 1 mM GSH, 0.75 mM | Buffer 3 plus 1 mM EDTA, 1 mM GSH, 0.75 mM |
| GSSG (18%) | GSSG, 1 mM PEG (27%) |
| Buffer 4 plus 1 mM EDTA, 2 mM GSH, 0.4 mM | Buffer 4 plus 1 mM EDTA, 2 mM GSH, 0.4 mM |
| GSSG | GSSG, 1 mM PEG (12%) |
| (18%) | |
| Buffer 5 plus 1 mM EDTA, 1 mM GSH, 0.75 mM | Buffer 5 plus 1 mM EDTA, 1 mM GSH, 0.75 mM |
| GSSG (13%) | GSSG, 1 mM PEG (15%) |
| Buffer 6 plus 1 mM EDTA, 1 mM GSH, 0.1 mM | Buffer 6 plus 1 mM EDTA, 1 mM GSH, 0.1 mM |
| GSSG | GSSG, 1 mM PEG (15%) |
| (9%) | |
| Buffer 7 plus 1 mM EDTA, 0.5 mM GSH, 0.5 mM | Buffer 7 plus 1 mM EDTA, 0.5 mM GSH, 0.5 mM |
| GSSG (6%) | GSSG (14%) |
| Buffer 8 plus 1 mM EDTA, 1 mM GSH, 0.2 mM | Buffer 8 plus 1 mM EDTA, 1 mM GSH, 0.2 mM |
| GSSG | GSSG, 1 mM PEG (31%) |
| (26%) | |
| Buffer 9 plus 1 mM EDTA, 1 mM GSH, 0.2 mM | Buffer 9 plus 1 mM EDTA, 1 mM GSH, 0.2 mM |
| GSSG | GSSG, 1 mM PEG (37%) |
| (29%) | |
| Buffer 10 (78%) | Buffer 10 with 1 mM PEG replacing glycerol |
| | (55%) |
| Buffer 11.1 mM EDTA and 4M urea, replaced | Buffer 11. With 1 mM EDTA and 6M GdnHCl, |
| with 3M urea on day 2, 2M urea on day 3, 1M | replaced with 5M GdnHCl on day 2, 4M GdnHCl |
| urea on day 4 and 0M urea on day 5 (63%) | on day 3, 3M GdnHCl on day 4, 2M GdnHCl on |
| | day 5, 1M GdnHCl on day 6 and 0M GdnHCl on |
| | day 7 (45%). |
| Buffer 12. 1 mM EDTA and 5 mM βME (59%) | Buffer 12. 1 mM EDTA, 5 mM βME, 20% |
| | glycerol (58%) |

Summary of Protein Refolding

The expression of ΔC-MxA protein in E. coli appears to be predominantly in the form of inclusion bodies. This provides a concentrated source of the protein for purification from E. coli. The protocol outlined above recovered ˜78% of the protein from these inclusion bodies in a soluble form, enabling the purification of ˜40 mg/L of culture. The procedure relies on the capturing of the protein onto a Co-based His-tag affinity resin and then refolding the protein into a soluble form by dialysis against a suitable buffer. The resin assists in this process, since it keeps the protein molecules physically separate and prevents aggregation. The ΔC-MxA protein may also be eluted from the column under denaturing conditions prior to refolding. Although the yields are slightly reduced (˜62% versus 78%) using this strategy, the protein purities are comparable (>95% by SDS PAGE). The composition of the dialysis buffer has an impact on the yields of refolded ΔC MxA protein; somewhat unexpectedly, the use of redox modulators such as GSH:GSSG and βME do not appear to promote efficient protein refolding, and glycerol rather than PEG was shown to be more effective at blocking aggregation during refolding.

No differences in protein characteristics or yields were observed using the T7 and T5 promoter systems.

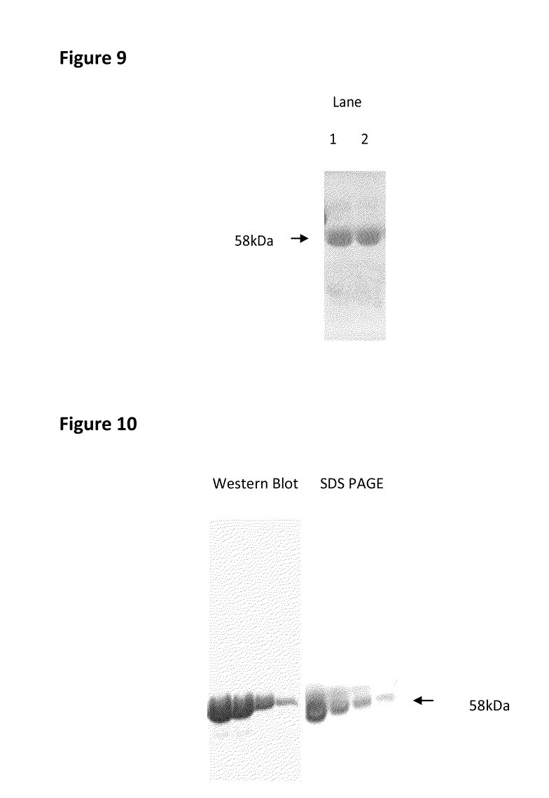

FIG. 9 shows an SDS PAGE comparison of calculated 10 μg of refolded ΔC-MxA protein produced from T7 (lane 1) and T5 (lane 2) promoter-driven expression constructs. Both constructs resulted in the expression of His-tag ΔC MxA protein as an inclusion body. Furthermore, using both expression systems, the disruption of the inclusion bodies with denaturant, capture of the recombinant material using a Talon Co-affinity resin and refolding by dialysis against 20 mM Tris, 100 mM KCl, 5 mM MgCl₂, 20% glycerol, 0.1% Nonidet P-40, pH 8.0 resulted in the purification of a protein with a molecular weight of 58 kDa and a relative purity of >95% based on SDS PAGE analysis.

FIG. 10 shows a Western and SDS PAGE analysis of the refolded ΔC-MxA protein produced using the T5 expression construct in BL21 (DE3). Protein (10 μg, 5 μg, 2.5 μg and 1 μg in lanes 1-4, respectively) was loaded, separated by SDS-PAGE (4-10% gradient) and transferred to a nylon membrane. The membrane was hybridized with mouse anti-His tag HRP conjugate and detected with KPL one substrate peroxidase reagent. The SDS PAGE gel indicates the protein is very pure and the visible product correlates with the His-Tag truncated MxA protein product. The strength of the Western signal correlates with the intensity of the Coomassie stained product on the SDS PAGE.

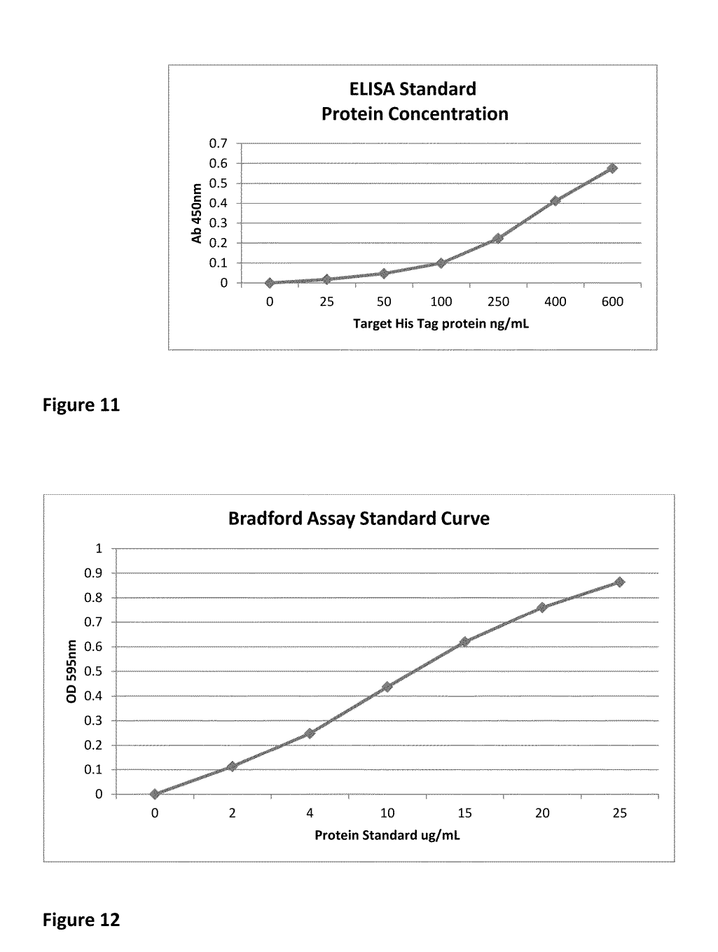

FIG. 11 shows quantification of the ΔC-MxA protein by ELISA. The same mouse-anti-His-tag HRP conjugate was used to quantify the recombinant material by ELISA. A standard protein Albumin-His tag was used as the standard and serial dilutions prepared from 0.001-1 mg/mL. The standard curve generated is shown in FIG. 12. Serial dilutions of the ΔC-MxA were generated in a similar way and also detected using the mouse-anti-His tag HRP conjugate. From this analysis, the protein concentration of the ΔC MxA protein was calculated to be: T5-generated=0.83±0.02 mg/mL; T7-generated=0.8±0.04 mg/mL.

FIG. 12 shows a Bradford assay determination of protein concentration. A Bradford assay standard curve was generated using a standard BSA solution. From this analysis, the protein concentration of the ΔC MxA preps was determined to be: T5-generated: 0.79 mg/mL; T7-generated: 0.8 mg/mL.

### Examples of Clinical Applications

The extremely large quantities of MxA protein that may be produced could be used in various therapeutic regimens. Also, in so-called “rational designing” of drugs, such quantities are needed in vitro as well as in vivo in the first stages of drug development.

The proteins that can be produced using the full length and truncated MxA constructs described herein may be used in any clinical application where functional MxA is needed. The truncated MxA protein may be more stable and create less immunogenic reactions, but is still functional. Therefore, it may be preferable for use in many clinical applications.

Since the MxA protein likely breaks down over time, it may be necessary to reinoculate the patient with multiple doses of MxA during the treatment period in order to maintain the protective properties of the MxA treatment. MxA may be administered by injection or transdermally through the skin, for example using a “patch”, for timed or slow release.

There is evidence that MxA may reduce cancer activity in cancer patients (see Mushinski et al., J. Biol. Chemistry, “Inhibition of Tumor Cell Motility by the Interferon-inducible GTPase MxA”, May 20, 2009, 284(22): 15206-15214, herein incorporated by reference). Recombinant MxA made using the constructs disclosed herein may be injected (or delivered transdermally through the skin using a patch) into cancer patients to counteract cancerous activity.

As another example, human MxA has antiviral properties that act against the influenza virus (see, for example, Horisberger, Am J Respir Grit Care Med., “Interferons, Mx genes, and resistance to Influenza Virus”, 152(4 Pt 2): S67-71, 1995, herein incorporated by reference). Recombinant MxA may be injected as an antidote and/or as protection against influenza. Using MxA can diagnose viral infection 1½ to 3 days before symptoms start. Once diagnosed, a patient could be given a dose of MxA. Then, when confronted with a pandemic, the patient will not get sick. Since the MxA protein likely breaks down over time, it may be necessary to reinoculate the patient with another dose of MxA in order to maintain the protective properties of the MxA treatment.

Interferon treatments are currently used to treat multiple sclerosis, but they are difficult to monitor. Studies have found that interferon induces MxA, and it is thought that monitoring of MxA is useful in reducing multiple sclerosis symptoms. MxA may be used in monitoring multiple sclerosis treatment to confirm how much active interferon (not neutralized by antibodies against interferon) is in a patient. Injection (or timed or slow release of the MxA transdermally through the skin using a patch) of recombinant MxA may help with symptoms and treatment of multiple sclerosis.

Another use for recombinant MxA made using the constructs described herein is for animal diagnostics and transport. There is a lot of concern when transporting animals that they will encounter and potentially contract a number of viral infections. Injecting (or transdermally delivering using a patch) an animal with MxA before transport could decrease or eliminate the requirement for quarantine by protecting the animal from the viral infections they could come into contact with during transport.

Another use for recombinant MxA made using the constructs described herein is for human and animal therapeutics. The natural activity of MxA protein blocks the eventual replication of virus using the host's cellular components and machinery.

The proteins produced from the truncated MxA constructs would be especially useful in the applications above, since they have longer storage stability than the proteins produced from the full length MxA constructs.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A control preparation for testing the quality of MxA protein comprising a C-terminal truncated human MxA protein that retains biological activity of an MxA protein.

2. The control preparation of claim 1, wherein the C-terminal truncated human MxA protein is produced by a truncated MxA DNA construct driven by a promoter.

3. The control preparation of claim 2, wherein the truncated MxA DNA construct is selected from the group consisting of SEQ. ID. NO. 3 and SEQ. ID. NO. 16.

4. The control preparation of claim 2, wherein the promoter is selected from the group consisting of a T5 promoter and a T7 promoter.

5. The control preparation of claim 1, wherein the C-terminal truncated human MxA protein has an amino acid sequence of SEQ. ID. NO. 4.

6. The control preparation of claim 1, wherein the C-terminal truncated human MxA protein includes GTP binding areas.

7. The control preparation of claim 1, wherein the biological activity of the C-terminal truncated human MxA protein is equivalent to a biological activity of a full length MxA protein.

8. A human MxA construct that produces a C-terminal truncated human MxA protein that retains biological activity of the MxA protein, wherein the human MxA construct comprises a truncated MxA DNA sequence driven by a promoter.

9. The construct of claim 8, wherein the truncated MxA DNA sequence is selected from the group consisting of SEQ. ID. NO. 3 and SEQ. ID. NO. 16.

10. The construct of claim 8, wherein the promoter is selected from the group consisting of a T5 promoter and a T7 promoter.

11. The construct of claim 8, wherein the C-terminal truncated human MxA protein has an amino acid sequence of SEQ. ID. NO. 4.

12. The construct of claim 8, wherein the C-terminal truncated human MxA protein includes GTP binding areas.

13. The construct of claim 8, wherein the biological activity of the C-terminal truncated human MxA protein is equivalent to a biological activity of a full length MxA protein.

14. A human MxA construct that produces a full length human MxA protein, wherein the human MxA construct comprises a full length MxA DNA sequence driven by a T5 promoter.

15. The human MxA construct of claim 14, wherein the MxA DNA sequence is selected from the group consisting of SEQ. ID. NO. 1 and SEQ. ID. NO. 15.

16. The human MxA construct of claim 14, wherein the full length human MxA protein has an amino acid sequence of SEQ. ID. NO. 2.
